(19) United States
(12) Patent Application Publication
(10) Pub. No.: US 2014/0257240 A1
(43) Pub. Date: Sep. 11, 2014

(54) ARTICULABLE INTRODUCER SHEATH

(71) Applicant: Albert G. Burdulis, San Francisco, CA (US)

(72) Inventor: Albert G. Burdulis, San Francisco, CA (US)

(73) Assignee: Spinal Modulation, Inc., Menlo Park, CA (US)

(21) Appl. No.: 13/791,641

(22) Filed: Mar. 8, 2013

(51) Int. Cl. A61M 25/01; A61N 1/05

(52) U.S. Cl. CPC A61M 25/0147; A61N 1/0551
USPC 604/506; 604/158; 604/523; 606/129

(57) ABSTRACT

Articulable introducer sheaths and related methods and systems for accessing various surgical sites are disclosed. An introducer sheath comprises a tubular body and a filament. The tubular body has a proximal portion, a distal portion, and a central lumen. The filament is constrained on or within at least a portion of the proximal portion and unconstrained over at least a portion of the distal portion. The distal end of the filament is held or attached near a distal end of the tubular body so that advancement of the filament relative to the tubular body causes the unconstrained portion to bow out or extend radially outward, such as to engage a body tissue so as to steer the tubular body when the tubular body has been introduced into a surgical site, such as an epidural space. A surgical tool or medical implant can then be advanced through the sheath.

### BACKGROUND OF THE INVENTION

The present disclosure relates to medical devices, systems, and methods. In particular, the present disclosure relates to devices, systems, and methods for delivering surgical tools or medical implants, for example, one or more neuromodulation devices, such as stimulation electrode leads, or other similar devices, in the body of a patient. The devices, systems, and methods disclosed herein may find particular use for delivering devices and implants for the neuromodulation of the spinal anatomy but may also find use in laparoscopic surgery, catheter-based procedures, and many other minimally-invasive surgical techniques.

The application of specific electrical energy to the spinal cord for the purpose of managing pain has been actively practiced since the 1960s. The application of an electrical field to spinal nervous tissue can effectively mask certain types of pain transmitted from regions of the body associated with the stimulated nervous tissue. Such masking is known as paresthesia, a subjective sensation of numbness or tingling in the afflicted bodily regions. Such electrical stimulation of the spinal cord, once known as dorsal column stimulation, is also referred to as spinal cord stimulation or SCS.

FIGS. 1A-1B illustrate conventional placement of an SCS system 10. Conventional SCS systems typically include an implantable power source or implantable pulse generator (IPG) 12 and an implantable lead 14. Such IPGs 12 may be similar in size and weight to pacemakers and are typically implanted in the buttocks of a patient P, as shown, or in the abdominal wall, chest wall, or under the arm. Under fluoroscopy, the lead 14 can be implanted into the epidural space E or the spinal column and positioned against the dura layer D of the spinal cord S, as illustrated in FIG. 1B.

FIG. 2 illustrates example conventional paddle leads 16 and percutaneous leads 18. Paddle leads 16 typically have the form of a slab of silicon rubber having one or more electrodes 20 on its surface. Another example of a paddle lead 16 is illustrated in FIG. 3. Percutaneous leads 18 typically have the form of a tube or rod having one or more electrodes 20 extending therearound. Another example of a percutaneous lead 18 is illustrated in FIG. 4.

Paddle leads 16 and percutaneous leads 18 can be positioned within the epidural space E of the spinal column by different methods due to their size and shape. Percutaneous leads 18 can be positioned with the use of an epidural needle. Referring to FIG. 5, an epidural needle 22 can be inserted through the skin (not shown) and advanced between adjacent vertebrae V1, V2 so that it penetrates the epidural space. Thus, a conduit may be formed from outside of the body to the epidural space. The lead 18 can then be advanced through the needle 22 and into the epidural space. The lead 18 may typically be advanced in an antegrade fashion up the midline of the spinal column until it reaches the area of the spinal cord, that when electrically stimulated, produces a tingling sensation (parasthesia) that covers the patient's painful area. To locate this area, the lead can be moved and/or turned on and off while the patient provides feedback about stimulation coverage. Often, inadequate stimulation is obtained and the lead may be repositioned multiple times before adequate coverage is received. Because the patient participates in this operation and directs the operator to the correct area of the spinal cord, the procedure is performed under monitored anesthesia care.

Conventional paddle leads 16 are too large to fit through an epidural needle. Therefore, implantation of paddle leads 16 typically involves a mini laminotomy. A laminotomy is a neurosurgical procedure that removes part of a lamina of the vertebral arch. An incision is typically made slightly below the spinal cord segment to be stimulated. The laminotomy can create an opening 24 in the bone large enough to pass one or more paddle leads 16 through. FIG. 6 illustrates a mini laminotomy with a paddle lead 16 inserted therethrough so that the stimulating portion of the lead 16 resides against the dura layer D of the spinal cord S. The target area for stimulation usually has been located before this procedure during a spinal cord stimulation trial with percutaneous leads 18.

As with any surgery, surgical placement of stimulation leads is a serious procedure and should be treated as such. A variety of complications may result, including complications with the anesthesia medication, deep vein thrombosis (DVT), nerve damage, and infection, to name a few. Such complications may be exacerbated by the need to use an extraneous number of invasive surgical instruments or apply additional surgical steps.

Less invasive and more targeted treatment options have been developed. Such targeted treatment minimizes deleterious side effects, such as undesired motor responses or undesired stimulation of unaffected body regions which often occurs with conventional SCS. Thus, a target anatomy associated with a condition, such as pain, is directly neuromodulated while minimizing or excluding undesired neuromodulation of other anatomies. In some instances, such targeted neuromodulation includes neuromodulating the dorsal root ganglia, dorsal roots, dorsal root entry zones, or portions thereof while minimizing or excluding undesired effects on other tissues, such as surrounding or nearby tissues, portions of the ventral root and portions of the anatomy associated with body regions which are not targeted for treatment. Such neuromodulation is typically achieved by stimulation with the use of a lead having at least one electrode thereon. The lead is advanced through the patient anatomy so that the at least one electrode is positioned on or near the target.

In some instances, the lead is delivered to a target location with the use of a delivery system such as illustrated in FIGS. 7A-7D. Here, the delivery system includes a sheath 1122 (FIG. 7B), stylet 1124 (FIG. 7C) and introducing needle 1126 (FIG. 7D), and is further described and illustrated in US. patent application Ser. No. 12/687,737, entitled “Stimulation Leads, Delivery Systems and Methods of Use”, filed Jan. 14, 2010, and incorporated by reference for all purposes. In some embodiments, the sheath 1122 has a distal end 1128 which is pre-curved to have an angle α, wherein the angle α is in the range of approximately 80 to 165 degrees. The sheath 1122 is sized and configured to be advanced over the shaft 1103 of the lead 1100 until a portion of its distal end 1128 abuts the distal tip 1106 of the lead 1100. Thus, the ball shaped tip 1106 of this embodiment also prevents the sheath 1122 from extending thereover. Passage of the sheath 1122 over the lead 1100 causes the lead 1100 to bend in accordance with the precurvature of the sheath 1122. Thus, the sheath 1122 assists in steering the lead 1100 along the spinal column and toward a target DRG, such as in a lateral direction.

In at least some cases, a physician may encounter difficulty in steering a sheath, such as sheath 1122, or other instrument in a desired direction within the epidural space. This difficulty may be especially prevalent in patients that have had prior spine surgery. Part of the normal healing process of spine surgery involves the formation of scar tissue in the epidural space. The scar tissue can lead to post-operative pain, such as due to binding or tethering of a nerve root that is formed by the scar tissue. Pain can occur when the scar that is adhered to the nerve is stretched. Such pain often causes the need for further treatment. However, access to the desired locations within the epidural space for such treatment is often hindered due to the scar tissue and/or implanted devices resulting from the prior treatment. In some instances, patients have scar tissue adhesions without having prior spine surgery. This can occur from a bulging disc, a herniated disc or other injury. However, the results may be the same--limited access to anatomies targeted for treatment of pain.

A physician may also encounter difficulty in steering a sheath or other instrument in a desired direction within the epidural space when trying to treat anatomies that are difficult to reach, such as along the cervical spine. Typically, the epidural space is entered below the cervical region, such as at T1-T2, and any instruments are advanced from the entry point up 3-4 levels to the target location. Such a significant distance makes steering difficult, and the longer this distance, the more difficult steering becomes. Thus, it is desired to improve access to anatomies targeted for treatment, particularly in patients having challenging situations. Such procedures should be effective in treating pain and other ailments while minimizing complications, costs, and debilitation. At least some of these objectives will be met by the devices, systems, and methods of the present disclosure.

### SUMMARY OF THE INVENTION

Aspects of the present disclosure provide devices, systems, and methods for accessing and treating anatomies associated with a variety of conditions while minimizing possible complications and side effects.

An aspect of the disclosure provides an introducer sheath comprising a tubular body and a filament. The tubular body has a proximal portion, a distal portion, and a central lumen. The tubular body is configured for passage through a cannula. The filament is constrained on or within at least a portion of the proximal portion, unconstrained over at least a portion of the distal portion, and constrained near a distal end of the tubular body. Movement of the filament relative to the tubular body causes the unconstrained portion of the filament to extend radially outward from the tubular body so as to apply force to the distal end of the tubular body.

The cannula may comprise an epidural needle and the tubular body may be configured for advancement within an epidural space. In some embodiments, the application of force to the distal end of the tubular body repositions the distal end of the tubular body. It may be appreciated that movement of the filament can apply a force component to the distal end of the tubular body which assists in steering within the epidural space. Movement of the filament relative to the tubular body can cause the unconstrained portion of the filament to extend radially outward so that the filament contacts a wall of the epidural space. The tubular body may be configured for advancement within the epidural space due to sufficient stiffness. Extension radially outward of the filament may increase the stiffness.

The distal portion of the tubular body may have a pre-curvature. Typically, the pre-curvature has an angle in the range of approximately 80 to 165 degrees. The unconstrained portion of the filament may be disposed along the pre-curvature so that the filament extends radially outward from an outside curvature of the pre-curvature.

The distal end of the filament may be removably attached near the distal end of the tubular body. The tubular body may have a side lumen extending at least partially through the tubular body and ending at a distal stop within which the filament is positioned. The filament can be removably withdrawn from the side lumen of the tubular body. The side lumen may have a lateral opening through which the unconstrained portion of the filament is configured to extend radially outward. The lateral opening may be disposed on the curved distal portion of the tubular body. The curvature may be curved in a first direction and the unconstrained portion of the filament may be configured to extend radially outward in a second direction opposite the first direction.

The tubular body may have a low profile of 13 to 14 gauge or greater. The tubular body may be comprised of polyimide or polyetheretherketone. The filament may be comprised of stainless steel, surgical steel, or nitinol.

The introducer sheath may further comprise a switch coupled to the proximal portion of the body and to the filament for articulating the filament between a radially extended configuration and a radially retracted configuration.

Another aspect of the disclosure provides an introducer sheath comprising a tubular body and a filament. The tubular body has a proximal portion, a distal portion and a central lumen. The tubular body is configured for passage through a cannula. The filament is constrained on or within at least a portion of the proximal portion and unconstrained over at least a portion of the distal portion. Movement of the filament relative to the tubular body causes the unconstrained portion of the filament to extend radially outward from the tubular body so as to engage a body tissue in a manner which assists in steering the tubular body within an area of a body. The body tissue may comprise a wall of an epidural space and the area of the body may comprise the epidural space.

A distal end of the filament may be removably attached near a distal end of the tubular body. The distal portion of the tubular body may have a curvature having an angle in the range of approximately 80 to 165 degrees. The tubular body may have a side lumen extending at least partially through the tubular body and ending at a distal stop. The filament may be positionable within the side lumen and can be removably withdrawn from the side lumen of the tubular body. The filament may be disposed within the side lumen of the tubular body and the distal stop may be configured to limit advancement of the filament relative to the tubular body. The side lumen may have a lateral opening through which the unconstrained portion of the filament is configured to extend radially outward. The lateral opening may be disposed on a curvature of the distal portion of the tubular body. The distal portion of the tubular body may be curved in a first direction and the unconstrained portion of the filament may be configured to extend radially outward in a second direction opposite the first direction.

The tubular body may have a stiffness of approximately 0.10 lbs·in² to approximately 0.50 lbs·in². The tubular body may have a low profile of 13 to 14 gauge or greater. The tubular body may be comprised of polyimide or polyetheretherketone. The filament may be comprised of stainless steel, surgical steel or nitinol.

The introducer sheath may further comprise a switch coupled to the proximal portion of the body and to the filament for articulating the filament between a radially extended configuration and a radially retracted configuration.

Yet another aspect of the disclosure provides a system for positioning an electrical stimulation lead near a target tissue. The system comprises one of the two introducer sheath described above and a lead. The lead comprises a shaft having at least one electrode disposed thereon. The lead is adapted to be advanced through the central lumen of the sheath.

The sheath may have an outer diameter which allows advancement through an introducer needle into a surgical space in a body of a patient and a stiffness which allows advancement along the surgical space to a position wherein the sheath can direct the lead toward the target tissue, and wherein withdrawal of the sheath positions the lead near the target tissue. The surgical space may comprise an epidural space of a spinal column of the patient and the target tissue may comprise a spinal nerve. The spinal nerve may comprise a dorsal root ganglion (DRG).

A further aspect of the invention provides a method for accessing a target tissue. An introducer sheath is inserted into a cannula accessing a surgical space in a body of a patient. The sheath is comprised of a tubular body having a proximal portion, a distal portion, and a central lumen. The sheath has a filament constrained on or within at least a portion of the proximal portion, unconstrained over at least a portion of the distal portion, and constrained near a distal end of the tubular body. The filament is moved relative to the tubular body so that the unconstrained portion of the filament extends radially outward from the tubular body so as to assist in guiding the distal end of the tubular body toward the target tissue.

The surgical space may comprise an epidural space of the patient and the target tissue. The target tissue may comprise a dorsal root ganglion. The filament may be moved so that the unconstrained portion contacts a wall of the epidural space.

The cannula may comprise an introducing needle having an inner diameter of less than or equal to approximately 0.067 inches. A lead may be placed into the sheath such that the lead extends out of the distal end of the sheath toward the target tissue. The sheath may be positioned within the surgical space so the lead extends out of the distal end of the sheath toward a dorsal root ganglion. The filament may be withdrawn from the tubular shaft before withdrawing the sheath from the surgical space.

Additional aspects and advantages of the disclosure will become readily apparent to those skilled in this art from the following detailed description, wherein only illustrative embodiments of the present disclosure are shown and described. As will be realized, the present disclosure is capable of other and different exemplary implementations, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A, 1B, 2, 3, 4, 5, 6 illustrate prior art;

FIG. 7A illustrates a currently known and used neurostimulation lead;

FIG. 7B illustrates a currently known and used introducer sheath;

FIG. 7C illustrates a currently known and used stylet for supporting the neurostimulation lead of FIG. 7A;

FIG. 7D illustrates a currently known and used introducer needle;

FIG. 8A illustrates an articulable sheath in an undeployed configuration according to an embodiment of the disclosure;

FIG. 8B illustrates the articulable sheath of FIG. 8A in a deployed configuration;

FIG. 9A illustrates a cross-section of the articulable sheath of FIG. 8A taken across line 9A in FIG. 8A;

FIG. 9B illustrates a cross-section of the articulable sheath of FIG. 8A taken across line 9B in FIG. 8A;

FIG. 9C illustrates a cross-section of the articulable sheath of FIG. 8A taken across line 9C in FIG. 8A;

FIG. 9D illustrates a cross-sectional view of the distal end of the articulable sheath of FIG. 9D;

FIGS. 10A and 10B illustrate the proximal end of an articulable sheath according to an embodiment of the disclosure;

FIG. 10C illustrates the proximal end of an articulable sheath according to another embodiment of the disclosure;

FIG. 11 illustrates a method of accessing an epidural space with the use of an introducing needle;

FIG. 12 illustrates a method of attaching a syringe to the needle of FIG. 11;

FIG. 13 illustrates a method of inserting an articulable sheath through the needle of FIG. 11 into the epidural space;

FIG. 14 illustrates an embodiment of the articulable sheath of the present disclosure advanced through a nerve root sleeve angulation from the epidural space, wherein the sheath is illustrated in an undeployed configuration (dashed line) and in at least partially deployed configurations (solid line); and

FIG. 15 illustrates an embodiment of the articulable sheath in a deployed configuration bracing against a wall of the epidural space.

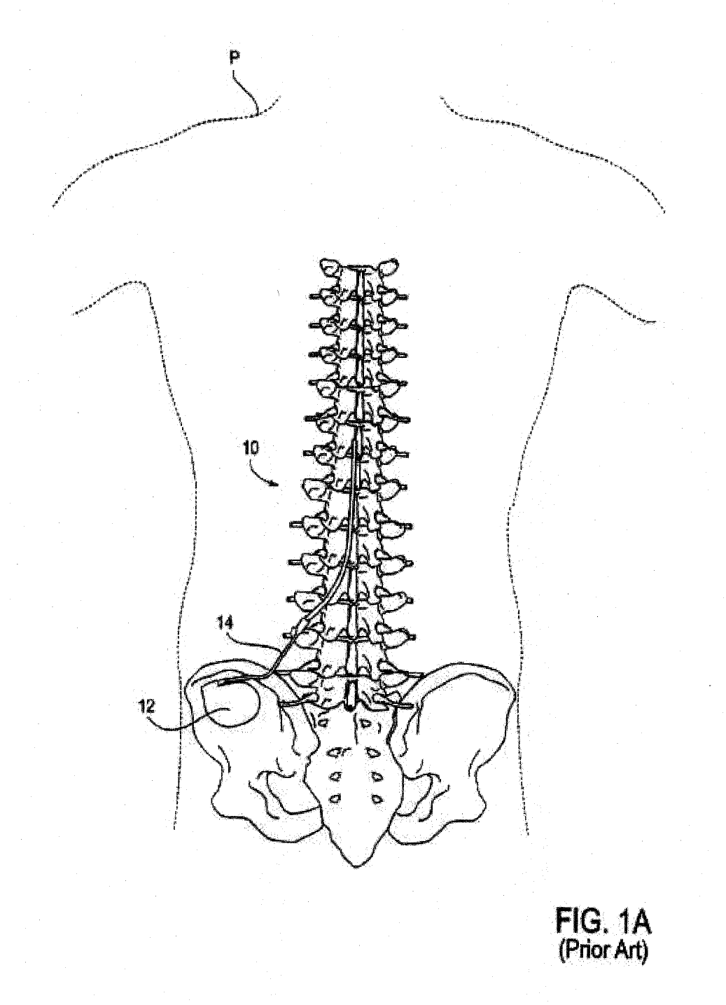

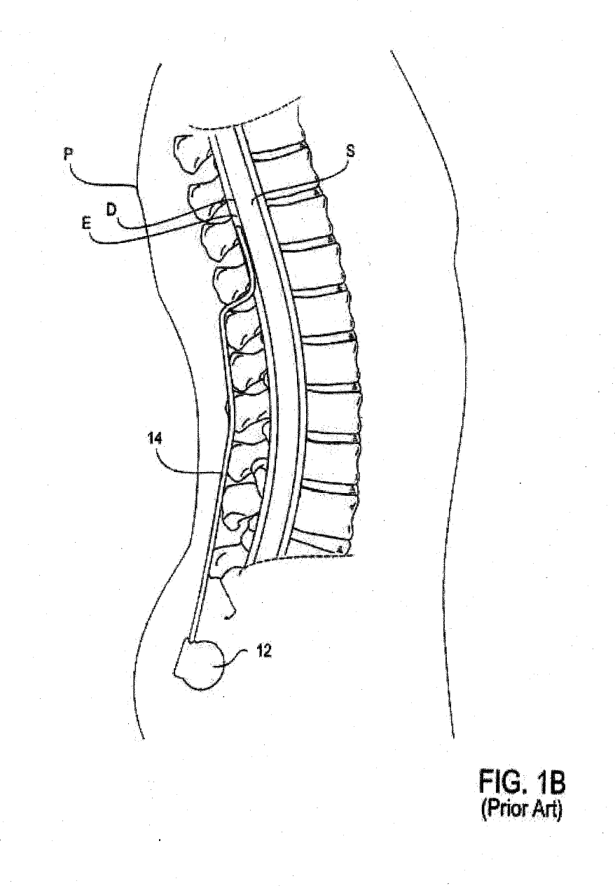

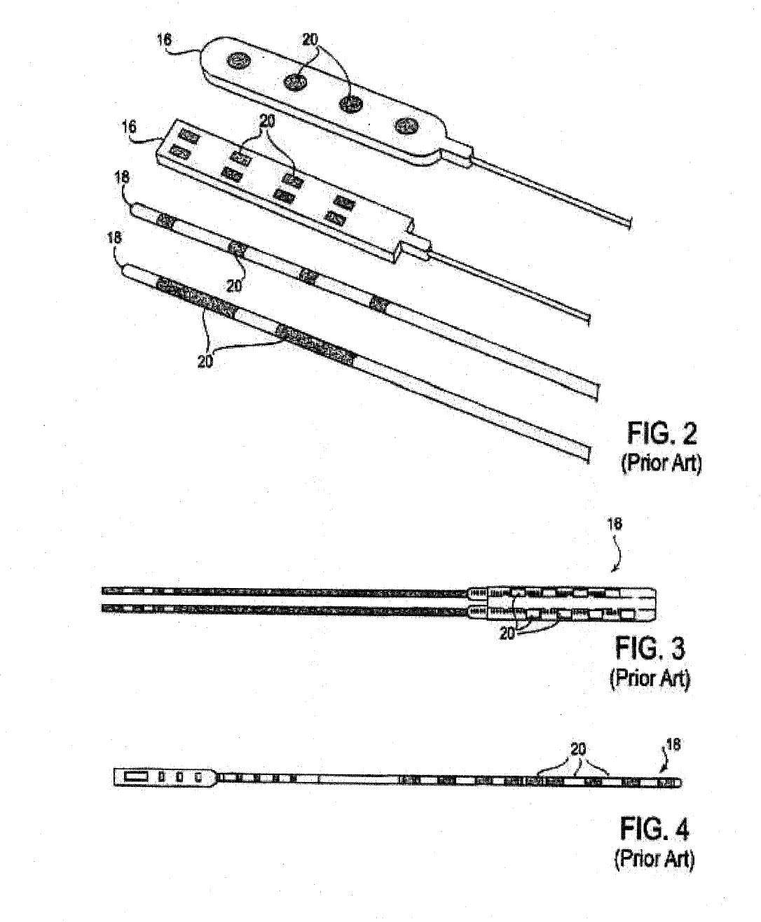

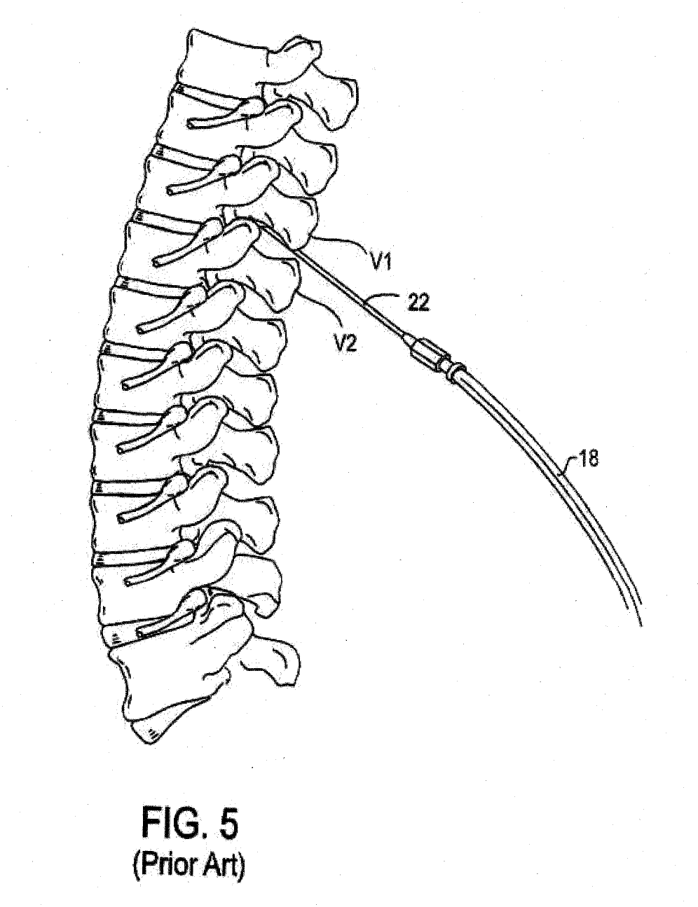

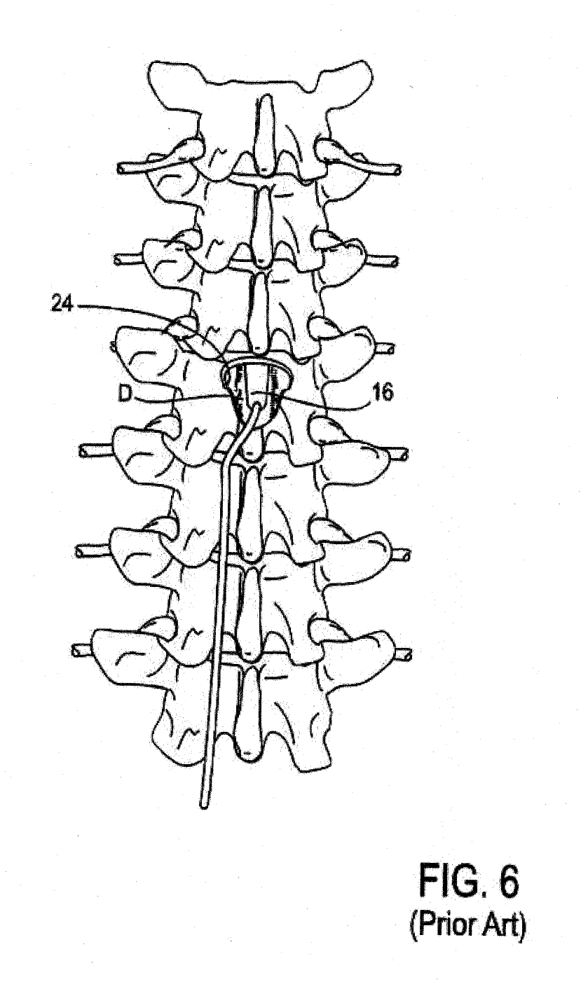

### DETAILED DESCRIPTION OF THE INVENTION

The present disclosure provides devices, systems, and methods for accessing target anatomies within the body, particularly target anatomies accessible through the epidural space. In the spine, the epidural space is the space within the spinal canal, between the vertebrae and the dura mater which covers the spinal cord. The epidural space contains lymphatics, loose fatty tissue, small arteries, and a network of large, thin-walled blood vessels called the epidural venous plexus. In some patients, the epidural space also contains scar tissue or fibrous lesions, such as due to prior surgery, a bulging disc, a herniated disc or other injury.

The present disclosure includes a delivery sheath for accessing target anatomies, particularly target anatomies accessible through the epidural space. The delivery sheath may be configured to be advanced within the epidural space and includes features which enhance stiffness or steerability when desired while maintaining ease of insertion through a cannula, such as an epidural needle. The delivery sheath may also include features which can assist in navigating around obstacles within the epidural space, such as scar tissue and adhesions, assist in directing the sheath in a lateral direction to access spinal anatomies, such as the dorsal root or dorsal root ganglion, and assist in advancing the sheath along multiple spinal levels to a target location distant from the entry site.

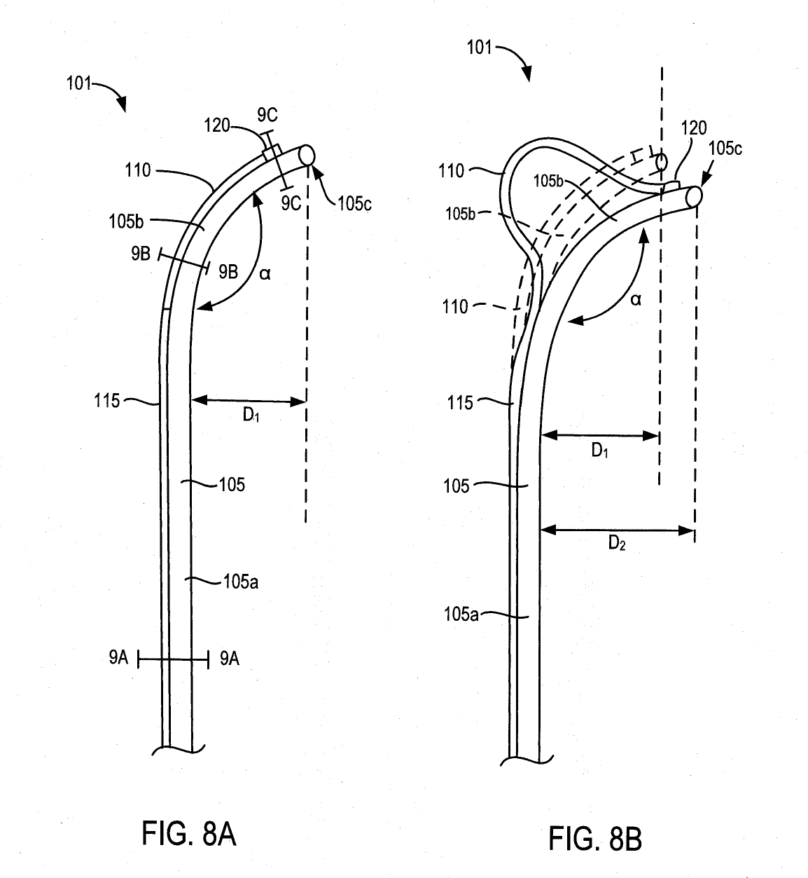

One embodiment of a delivery sheath 101 is illustrated in FIGS. 8A-8B. In this embodiment, the sheath 101 is articulable and is comprised of a tubular main body 105, a filament 110 positioned adjacent the tubular main body 105, a filament constraint 115, and a filament anchor 120. In this embodiment, the filament 110 extends within the filament constraint 115, along the proximal section 105a of the tubular main body 105, and terminates within the filament anchor 120 near the distal tip 105c of the tubular main body 105. Between the filament constraint 115 and the filament anchor 120, the filament 110 is exposed and unrestrained. It is in the area of the exposed region that the filament 110 may be deployed and able to extend outwardly away from the tubular main body 105. Typically, filament 110 is comprised of a flexible and/or resilient polymer or metal such as surgical steel, stainless steel, or nitinol. The material chosen may depend on the stiffness of the filament 110 desired. FIG. 8A illustrates the sheath 101 in an undeployed configuration wherein the filament 110 is not extended and resides adjacent to the main body 105 along its length. And, FIG. 8B illustrates the sheath 101 in the deployed configuration wherein the filament 110 extends outwardly away from the main body 105 in the area of the filament 110 that is not restrained. Thus, the filament 110 may be able to form a loop or curved shape. Deployment can be achieved by advancing the filament 110 within the filament constraint 115. Since the filament 110 may be restrained within the filament constraint 115 and fixed within the filament anchor 120, such advancement can causes the filament 110 to bow or extend radially outwardly in free space between the constraint 115 and anchor 120. This radial extension of the filament 110 provides a variety of advantages, such as increased stiffness, increased steerability, and leverage to push or direct the sheath 101 in a desired direction, such as toward a target tissue structure. For example in some embodiments, as illustrated in FIG. 8B, such radial extension of the filament 110 applies a force component to the anchor 120 and thus the distal end of the sheath 101. This causes the main tubular body 105 to bend in the direction of the force component, such as distally. Such features will be described in more detail below.

The main tubular body 105, the filament constraint 115, and the filament anchor 120 may be integral with one another. For example, the filament constraint 115 and the filament anchor 120 may be formed in the main tubular body 105, such as lumens within a wall of the tubular body 105. In such an example, the filament 110 may be fixed within the lumen of the filament anchor 120, such as with adhesive. In some embodiments, the filament constraint 115 and filament anchor 120 are formed from a single lumen extending along the tubular body 105 and a wall of the lumen is skived or cut away to create the exposed region. It may be appreciated that in some embodiments the filament constraint and tubular body 105 are integral and the filament anchor 120 is not integral. For example in some embodiments, the filament anchor 120 is simply fixed to an exterior wall of the tubular body 105, such as by adhesive and/or a crimping ring. It may also be appreciated that in some embodiments, one or more of the filament constraint 115 and filament anchor 120 are not integral with the tubular body. For example, the filament constraint 115 may be comprised of a separate tube or series of restraints attached to the tubular body 105 where the filament passes therethrough. In any case, overall, the articulable sheath 101 will typically have a relatively low profile, for example, of 13 to 14 gauge or greater. Typically, the sheath 101 is configured to be advanced through a 12 to 16 gauge needle, particularly a 14 gauge needle.

In the embodiment of FIGS. 8A-8B, the tubular main body 105 comprises a straight proximal section 105a and a curved distal section 105b, which is pre-curved to have an angle α. In some embodiments, the angle α is in the range of approximately 15 to 165 degrees. Any suitable angle, however, may be used. The bend can also be characterized by the lateral distance D1 from the distal tip 105c of the sheath 101 to the outer surface of the straight proximal section 105a of the tubular main body 105. In some embodiments, the distance D1 is approximately 0.030-0.375 inches. The sheath 101 may be sized and shaped for particular types of delivery, such as antegrade, retrograde, and contralateral advancement within the epidural space, to name a few. In some embodiments, an antegrade sheath (configured for antegrade delivery) has a bend with an angle α of 90-110 degrees and a distance D1 of approximately 0.325-0.375 inches. Bends having an angle α less than or equal to 150 degrees and a distance D1 of greater than or equal to 0.225 inches typically improve the ease of delivery when using an antegrade approach to an anatomical target such as the dorsal root ganglion (DRG). In some embodiments, an alternative sheath (configured for retrograde or contralateral delivery) has a bend with an angle α of approximately 130-150 degrees and a distance D1 of approximately 0.045-0.095 inches. Bends having an angle α less than or equal to 165 degrees and a distance D1 of greater than or equal to 0.030 inches typically improve the ease of delivery when using a retrograde or contralateral approach to a target such as the DRG. In some embodiments, the entire tubular main body 105 is straight and the sheath 101 relies on the filament 110 to steer or bend the sheath 101.

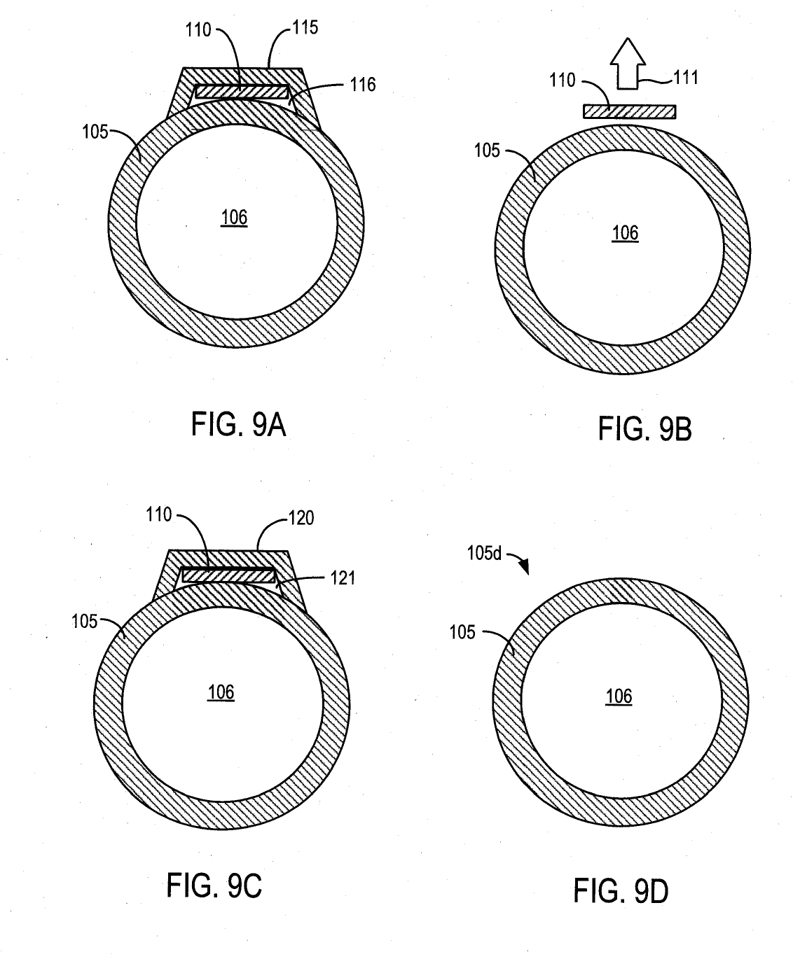

FIGS. 9A-9D illustrate cross-sections of the embodiment of the articulable sheath 101 of FIG. 8A. FIG. 9A shows a cross-section of the straight proximal portion 105a of the sheath 101, taken across line 9A in FIG. 8A. As shown in FIG. 9A, a central lumen 106 is disposed within the tubular main body 105. A variety of medical tools, such as catheters and electrical stimulation leads, may be passed through the central lumen 106. The filament constraint 115 is coupled with, and will typically be integral with, the main tubular body 105. A side lumen 116 is defined by the filament constraint 115 and the outer wall of the tubular main body 105. The filament 110 is housed within the side lumen 116.

FIG. 9B shows a cross-section of the curved distal portion 105b of the sheath 101, taken across line 9B in FIG. 8A. At the curved distal portion 105b of the sheath 101, the filament 101 is disposed adjacent to the outer wall of the main tubular body 105 and is not constrained by the filament constraint 115. Because the distal end of the filament 101 is held in place or at least limited in movement in the distal direction, advancement of the filament 101 in the distal direction will cause the filament 101 to extend radially outward or bow out in a direction indicated by arrow 110.

FIG. 9C shows a cross-section of the portion of the sheath 101 at the anchor 120, taken across line 9C in FIG. 8A. The anchor 120 is coupled with, and will typically be integral with, the main tubular body 105. A cavity 121 is defined by the anchor 120 and the outer wall of the tubular main body 105. The distal most portion of the filament 110 is housed within the cavity 121.

FIG. 9D shows a cross-sectional view of the distal tip 105c of the articulable sheath 101. In this embodiment, the filament 110, the filament constraint 115, and the filament anchor 120 are not present at this cross-section. However, it may be appreciated that in some embodiments, the filament and/or filament constraint may reach the most distal tip of the sheath 101. The distal tip 105c may be rounded or otherwise atraumatic to facilitate safe insertion and advancement in a surgical site.

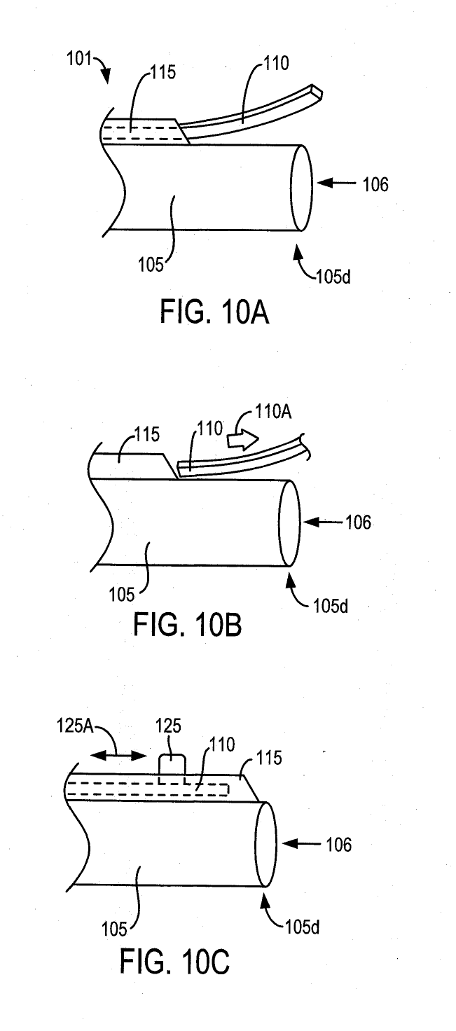

FIGS. 10A and 10B show an embodiment of the proximal end 105d of the articulable sheath 101. In many embodiments, the filament constraint 115 ends distal of the proximal end 105d of the articulable sheath 101. In such embodiments, the proximal end of the filament 110 extends out of the proximal end of the filament constraint 115 where it can be hand-manipulated by a user or operator. For example, the user or operator may advance the filament 110 forward to cause the unconstrained portion of the filament 110 to bow out or extend radially outward as shown in FIG. 8B. Or, the user or operator may retract the filament 110 proximally in a direction 110A to un-bow or reduce bowing of the unconstrained portion, returning the unconstrained portion of the filament 110 to a position adjacent the main tubular body 105. Further retraction of the filament 101 completely removes the filament from the sheath 101. Such complete retraction may be useful in a variety of circumstances, particularly when tissue or an obstruction has undesirably entered the space between the radially extended filament 110 and the tubular main body 105. In such instances, entrapment of tissue or an obstruction may make movement, steering or removal of the sheath 101 more challenging. Therefore, in some embodiments, the filament 110 is removeable entirely to free the entrapment. In some embodiments, the filament 110 can then be reintroduced to the sheath 101 for use as described above. Such reintroduction can be achieved while the sheath 101 is positioned in the body or the sheath can be removed for such reintroduction of the filament and then the sheath is repositioned in the body.

FIG. 10C shows the proximal end 105d of an articulable sheath 101 according to another embodiment of the disclosure. In this embodiment, the filament constraint 115 is not open at its proximal end and the proximal portion of the filament 101 is coupled to an articulable control mechanism or knob 125 that can be articulated by the user to move the filament 110 distally and proximally to respectively deploy or un-deploy the articulable sheath 101.

The tubular main body 105, the filament constraint 115, and/or the filament anchor 120 may be made of a relatively rigid polymeric material such as polyimide or polyetheretherketone (PEEK). In preferred embodiments, one or more of the components 105, 115, 120 are comprised of a plastic material, such as a thermoset and/or thermoplastic material. Polyimide may be preferred due to the thinness of its walls while retaining high strength, superior shape memory and shape retention. Polyimide can also be straightened for passage through an introducing cannula, such as the introducing needle 126 described below, without kinking In some embodiments, the sheath 101 is comprised of polyimide material having a wall thickness in the range of approximately 0.002-0.006, more particularly approximately 0.003-0.006 inches. It may be appreciated that other materials may be used provided the resulting sheath has an appropriate stiffness to allow advancement along the epidural space, while having a wall-thickness thin enough to allow passage of the sheath and a medical implant or surgical tool through an introducing needle to the epidural space, and while having a sufficiently low coefficient of friction to allow desirable passage of the medical implant or surgical tool therethrough. Further, the resulting sheath should be kink-resistant and formable into a desired shape. Examples of other materials potentially meeting these criteria include nylon, polycarbonate, acrylonitrile butadiene styrene (ABS), Polyethylene terephthalate (PET) and Pebax, to name a few.

In some embodiments, the tubular main body 105, the filament constraint 115, and/or the filament anchor 120 is comprised of a single stiffness or unidurometer material, especially where the sheath 101 is introduced to the epidural space together with a stylet or other device pre-loaded, thereby sharing the delivery workload. In particular, when the stylet or other device substantially fills the inner lumen 106 of the tubular main body 105, strength and kink resistance can be bolstered for delivery robustness. By contrast, if the sheath 101 were introduced alone, stiffness transitions, such as durometer/materials changes, or reinforcements, such as braiding, may be desired for kink resistance. However, it may be appreciated that sheath 101 may optionally be comprised of a reinforced polymer, such as a braided polymer, or may be comprised of a construct of various materials. For example, the tip 105c of the sheath 101 may be comprised of a differing material or a thinner material to create a less traumatic or an atraumatic tip. Such a tip may be more flexible than the remainder of the sheath which provides increased torqueability and pushability. Further, it may be appreciated that the sheath 101 may optionally be comprised of a flexible metal or metal/polymer construct.

In some embodiments, the sheath 101 has a stiffness of approximately 0.10 lbs·in² to approximately 0.50 lbs·in², particularly approximately 0.10 lbs·in² to approximately 0.30 lbs·in², more particularly approximately 0.14 lbs·in² to approximately 0.25 lbs, more particularly, 0.17-0.23 lbs·in². The stiffness of the sheath 101 may be enhanced by advancement of the filament 110. For example, in some embodiments, the sheath 101 has an overall stiffness which is desirable for advancement through a needle into the epidural space. The sheath 101 is then advanced along the epidural space, such as along a plurality of spinal levels. In some embodiments, the sheath 101 is advanced along one, two, three, four, five, six or more spinal levels. The more spinal levels the sheath 101 is advanced along, the more stiffness may be desired to maintain position of the sheath 101, such as along the midline, or to steer the sheath 101 in a desired direction. An example of such steering includes directing the distal tip of the sheath 101 in a lateral direction, away from the midline, such as toward a dorsal root or dorsal root ganglion. Stiffness of the sheath 101 can be increased in vivo by deployment of the sheath 101 (i.e. advancement of the filament 110 so that it bows radially outwardly creating a bump or loop shape). The degree of stiffness can be varied by varying the advancement of the filament 110. In some embodiments, advancement of the filament 110 applies a force component to the filament anchor 120, and therefore distal end of the sheath 101, which provides added stiffness to the sheath 101 when advancing the sheath 101 along various anatomies. For example, when accessing a dorsal root ganglion, the sheath 101 may be advanced within the epidural space toward the foramen containing the dorsal root ganglion. Upon approach to the dorsal root ganglion, the sheath 101 may encounter resistance by the foramen. Deployment of the filament 110 may provide the desired increase in stiffness to enter the foreman for positioning the distal end of the sheath 110 close to, adjacent or within the foramen. Such deployment may also bend the distal end of the sheath 101, such as illustrated in FIG. 8B, for added guidance. As mentioned previously, in some embodiments, the sheath 101 is pre-curved to have an angle α and such a bend can be characterized by the lateral distance D1 from the distal tip 105c of the sheath 101 to the outer surface of the straight proximal section 105a of the tubular main body 105, as illustrated in FIG. 8A and in dashed line in FIG. 8B. Advancement of the filament 110 additionally bends the sheath 101, as shown in solid line in FIG. 8B, so as to increase the lateral distance D2 from the distal tip 105c of the sheath 101 to the outer surface of the straight proximal section 105a of the tubular main body while maintaining the original pre-curve angle α. Thus, the deployed sheath 101 of FIG. 8B has a compound curvature. The point of bending due to the deployment of the filament is dependent on the location of the unrestrained space along the sheath 101. Thus, in some embodiments, the point of bending due to deployment matches the pre-curvature and in some embodiments the point of bending due to deployment is different and therefore creates a compound curvature.

The above described articulable sheath 101 can be used to deliver medical instruments, tools, implants, catheters, leads, and the like through the anatomy of a patient toward a target site. For example, the sheath 101 may be used to deliver an electrical stimulation lead to a target dorsal root ganglion (DRG). Thus, embodiments of epidural delivery methods are described herein. In particular, such embodiments are described and illustrated as an antegrade approach. It may be appreciated that, alternatively, the devices and systems of the present invention may be used with a retrograde approach or a contralateral approach. Likewise, at least some of the devices and systems may be used with a transforaminal approach, wherein the DRG is approached from outside of the spinal column. Further, the target DRG may be approached through the sacral hiatus or through a bony structure such as a pedicle, lamina, or other structure. The target DRG may be any DRG along the spinal column, including the cervical spine, thoracic spine, lumbar spine, and sacral spine. The sheath 101 may find particular use for delivering a catheter or lead in the cervical spine where tissue structures are relatively small and difficult to navigate, typically involving an entry point at a distance from the target treatment location. While the delivery of a lead or other implant to the spinal column or nearby nerve structures is described, the articulable sheath of the present disclosure may be used for other anatomies and purposes such as for laparoscopic surgery, catheter-based vascular procedures, and other minimally invasive surgical techniques.

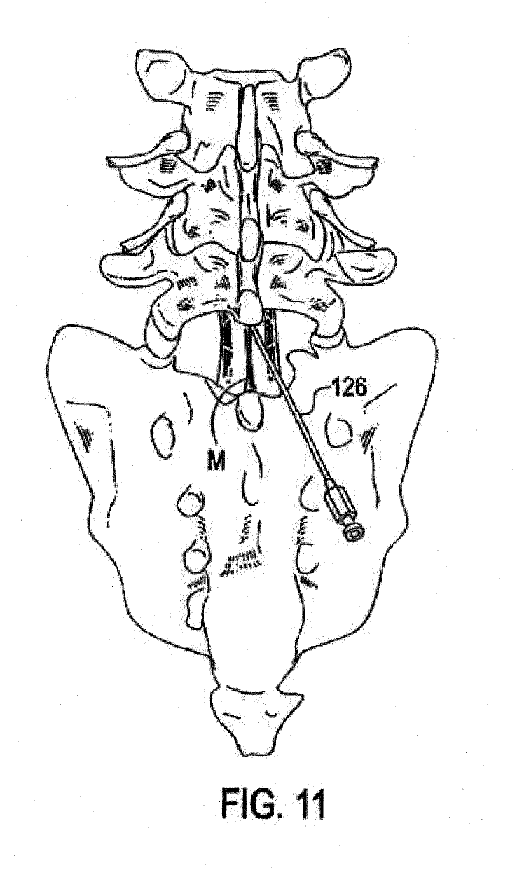

Epidural delivery involves accessing the epidural space. The epidural space can be accessed with the use of an introducing needle 126, as illustrated in FIG. 11. The introducing needle 126 may be a standard epidural access device used commonly with an anti-coring stylet. Such needles 126 are typically comprised of stainless steel and have an atraumatic tip to prevent insertion through the spinal dural sac. In some embodiments, the introducing needle is a 14 gauge thin-wall; however, it may be appreciated that other sized needles may be used, particularly smaller diameter needles. Typically, the skin is infiltrated with local anesthetic such as lidocaine over the identified portion of the epidural space. The insertion point is usually near the midline M, although other approaches may be employed. Typically, the needle 126 is inserted to the ligamentum flavum and a loss of resistance to injection technique is used to identify the epidural space.

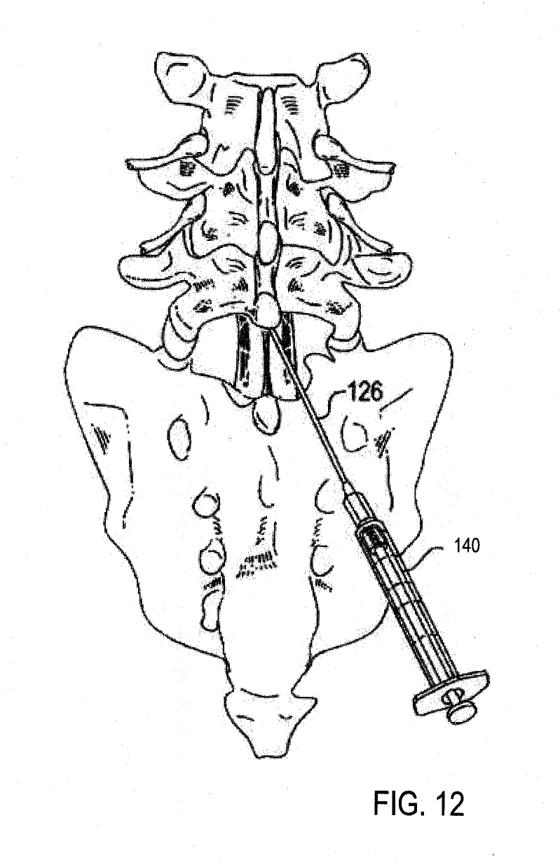

Referring to FIG. 12, a syringe 140 is then attached to the needle 126. The syringe 140 may contain air or saline. Traditionally either air or saline has been used for identifying the epidural space, depending on personal preference. When the tip of the needle 126 enters a space of negative or neutral pressure (such as the epidural space), there will be a “loss of resistance” and it will be possible to inject through the syringe 140. At that point, there is now a high likelihood that the tip of the needle 126 has entered the epidural space. Further, a sensation of “pop” or “click” may be felt as the needle breaches the ligamentum flavum just before entering the epidural space. In addition to the loss of resistance technique, real-time observation of the advancing needle 126 may be achieved with a portable ultrasound scanner or with fluoroscopy. Likewise, a guidewire may be advanced through the needle 126 and observed within the epidural space with the use of fluoroscopy.

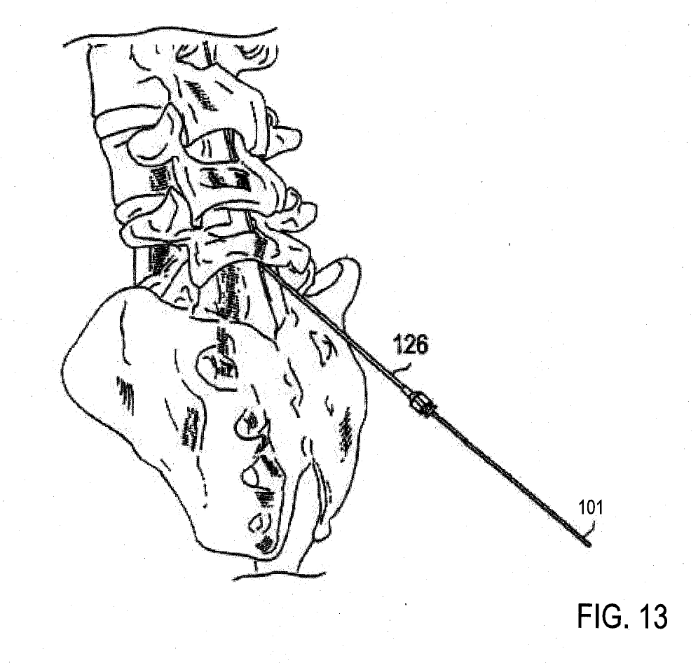

Once the needle 126 has been successfully inserted into the epidural space, the syringe 140 is removed. The sheath 101 can then be inserted through the needle 126, into the epidural space, as illustrated in FIG. 13. The sheath 101 may be loaded with a catheter, an electrical lead, or any other surgical tool, medical implant, or the like.

The sheath 101 and any other surgical tool, medical implant, or the like will typically be passable through the needle 126 for introduction to the epidural space without damage to the needle 126 or to the devices passed therethrough. Thus, access can be achieved through a single entry point and the devices can be advanced, retracted, removed, and reinserted through the needle 126 with ease and without irritation, injury, or disruption to the tissues surrounding the entry point.

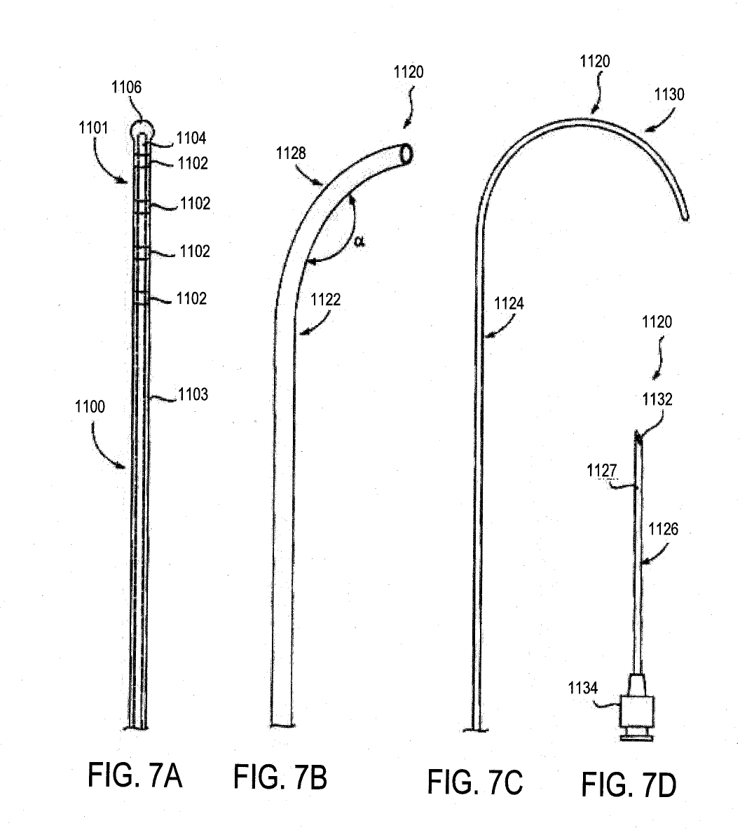

In some embodiments, the sheath 101 is used with a lead 1101 and stylet 1124, such as illustrated in FIGS. 7A and 7C respectively, and further described and illustrated in U.S. patent application Ser. No. 12/687,737, entitled “Stimulation Leads, Delivery Systems and Methods of Use”, filed Jan. 14, 2010, and incorporated by reference for all purposes. In such situations, the sheath 101 of the present invention is used instead of the sheath 1122. Thus, the sheath 101 is sized and configured to be advanced over the shaft 1103 of the lead 1100 until a portion of its distal end 128 abuts the distal tip 1106 of the lead 1100. The ball shaped tip 1106 of the lead 1100 also prevents the sheath 101 from extending thereover. Passage of the sheath 101 over the lead 1100 causes the lead 1100 to bend in accordance with the precurvature of the sheath 101. Thus, the sheath 101 assists in steering the lead 1100 along the spinal column and toward a target DRG, such as in a lateral direction.

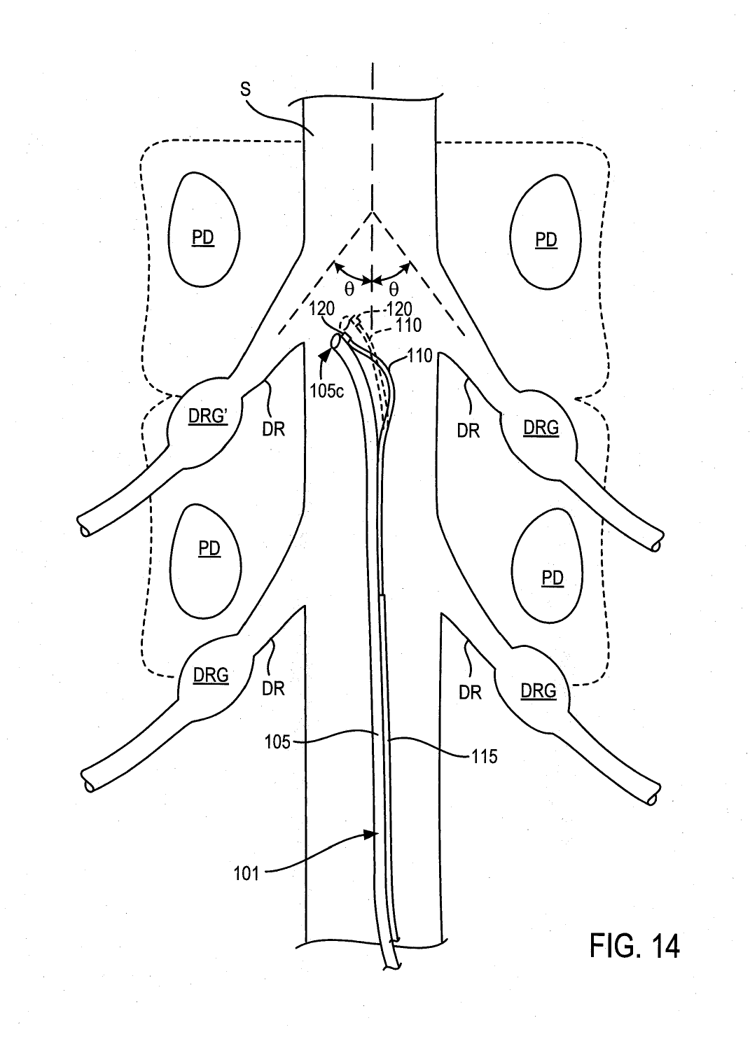

FIG. 14 illustrates example positioning of the sheath 101 within the epidural space to access a target dorsal root ganglion DRG′, such as when used with the lead 1101 and stylet 1124 as mentioned above. Here, the spinal cord and the pairs of nerves along the spinal cord, which are known as spinal nerves, are shown. The spinal nerves include both dorsal and ventral roots which fuse in the intravertebral foramen to create a mixed nerve which is part of the peripheral nervous system. At least one dorsal root ganglion (DRG) is disposed along each dorsal root prior to the point of mixing. Thus, the neural tissue of the central nervous system can be considered to include the dorsal root ganglions and exclude the portion of the nervous system beyond the dorsal root ganglions, such as the mixed nerves of the peripheral nervous system.

Accessing the dorsal root ganglions can be challenging, particularly from an antegrade epidural approach. As shown, each DRG is disposed along a dorsal root DR and typically resides at least partially between the pedicles PD or within a foramen. Each dorsal root DR exits the spinal cord S at an angle θ. This angle θ is considered the nerve root sleeve angulation and varies slightly by patient and by location along the spinal column. The average nerve root angulation in the lumbar spine is significantly less than 90 degrees and typically less than 45 degrees. Therefore, accessing this anatomy from an antegrade approach may involve making a sharp turn through, along or near the nerve root sleeve angulation. It may be appreciated that such a turn may follow the nerve root sleeve angulation precisely or may follow various curves in the vicinity of the nerve root sleeve angulation.

FIG. 14 illustrates an embodiment of the sheath 101 of the present disclosure positioned so as to access the target DRG′. The sheath 101 is inserted epidurally and advanced in an antegrade direction along the spinal cord S within the epidural space. In this embodiment, the sheath 101 is advanced along more than one spinal level to reach the target DRG′. While the sheath 101 in the undeployed configuration may be curved, it may be difficult to reach some target tissue structures in at least some cases. For example, traversing the distance between the epidural entry point and the target DRG′ may be challenging due to lack of stiffness. Likewise, making an optimal bend in a lateral direction, away from the midline of the spinal cord, may be challenging. Additionally or alternatively, the sheath 101 may find some resistance when approaching and/or entering the foramen surrounding the target DRG′. Each of these challenges may be improved or overcome with at least partial deployment of the sheath 101, i.e. by advancing the filament 110 as described above to cause the filament 110 to bow out or extend radially outward. In some embodiments, the filament 110 is deployable to any desired degree, wherein partial deployment or full deployment may be utilized. Thus, in some embodiments, the further the deployment, the more stiffness or steering is imparted. In this embodiment, deployment provides increased stiffness to the sheath 101 and guides the distal tip of the sheath 101 toward the target DRG. Thus, FIG. 14 illustrates the sheath 101 in the undeployed position (dashed line) and then in the deployed position (solid line) where the distal tip of the sheath 101 has been repositioned.

Once positioned one or more a leads, catheters, tools or devices can be advanced through the inner lumen 106 of the sheath 101 and delivered to the target anatomy, such as the target DRG′. It may be appreciated that one or more of these devices may be pre-loaded in the sheath 101, such as the lead 1101 and stylet 1124 described above. In such instances, the sheath 101 may be deployed with the one or more devices therein, wherein the one or more devices are repositioned along with the sheath 101 during deployment. Thus, the one or more devices are steered toward the target DRG′ along with the distal end of the sheath 101.

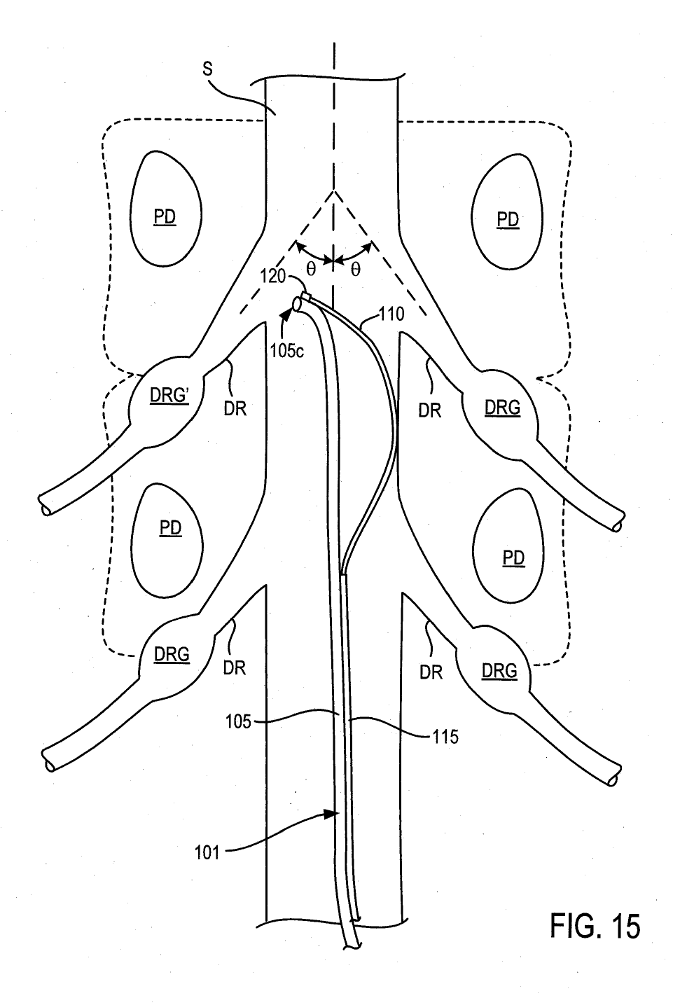

FIG. 15 illustrates another embodiment of the sheath 101 of the present disclosure positioned so as to access the target DRG′. The sheath is inserted epidurally and advanced in an antegrade direction along the spinal cord S within the epidural space. In this embodiment, the sheath 101 is advanced along more than one spinal level to reach the target DRG. While the sheath 101 in the undeployed configuration may be curved, it may be difficult to reach some target tissue structures in at least some cases, as mentioned above. Each of the challenges may be improved or overcome with deployment of the sheath 101, i.e. advancing the filament 110 as described above to cause the filament 110 to bow out or extend radially outward. In this embodiment, the filament 110 is deployed so that the filament resides against a body tissue, such as the opposite side of the epidural space, as illustrated in FIG. 15. Thus, the deployed filament serves as a “back stop” for the sheath 101, bracing the sheath 101 against a portion of the anatomy that provides resistance and support. This in turn applies a force component to the filament anchor 120, and therefore distal end of the sheath 101, which provides added stiffness and steerability to the sheath 101. Such deployment may also bend the distal end of the sheath 101 for added guidance. It may be appreciated that the filament may be braced against any suitable portion of the anatomy, including a bony structure, a pedicle, a wall of the epidural space, scar tissue, a blood vessel, a lymphatic vessel, fatty tissue, or a combination of these, to name a few.

Once positioned, one or more a leads, catheters, tools or devices can be advanced through the inner lumen 106 of the sheath 101 and delivered to the target anatomy, such as the target DRG. It may be appreciated that one or more of these devices may be pre-loaded in the sheath 101, such as the lead 101 and stylet 124 described above. In such instances, the sheath 101 may be deployed with the one or more devices therein, wherein the one or more devices are repositioned along with the sheath 101 during deployment. Thus, the one or more devices are steered toward the target DRG′ along with the distal end of the sheath 101.

It may be appreciated that the invention of the present disclosure may be used in the treatment of a variety of anatomies associated with a variety of conditions, particularly conditions that are associated with or influenced by the nervous system. Examples of such conditions include pain, itching, Parkinson's Disease, Multiple Sclerosis, movement disorders, spinal cord injury, asthma, chronic heart failure, obesity and stroke (particularly acute ischemia), to name a few. The devices, systems, and methods of the present disclosure can be used to deliver treatment devices to difficult-to-access locations in a patient's anatomy. The devices, systems, and methods of the present disclosure as described herein are used to deliver an implant or device, an electrode lead for neurostimulation in particular, to a target DRG but may also be used for laparoscopic surgery, catheter-based procedures, and many other minimally-invasive surgical techniques.

It may be appreciated that the articulable sheaths of the present disclosure may be substantially straight or have one or more curves at a variety of angles. The desired curvature may depend on a variety of factors, including the approach used (such as antegrade, retrograde, or contralateral), the choice of target anatomy, and the particular anatomical features of the individual patient, to name a few.

Typically, the systems and devices can be used to access and deliver stimulation electrode leads to various portions of neural tissue of the central nervous system, which includes the spinal cord and the pairs of nerves along the spinal cord which are known as spinal nerves. In some embodiments, the systems and devices of the present invention may be used to access and deliver devices, such as stimulation electrode leads, to one or more dorsal root ganglia, dorsal roots, dorsal root entry zones, or portions thereof. However, it may be appreciated that other anatomies may be targeted for treatment.

It may further be appreciated that when an articulable sheath is described as directing a surgical tool or medical implant toward a target anatomy, such direction may be in the general vicinity of the target anatomy allowing for additional steps to direct the surgical tool or medical implant even closer toward the target anatomy. For example, a curved sheath may direct the medical implant or surgical tool away from the midline of the spinal column, toward a target DRG. However, straight advancement of the lead therefrom may be below, above, or not desirably close enough to the target DRG. Therefore, the filament of the articulable sheath may be deployed to varying degrees to position the surgical tool or medical implant closer to the target DRG. For example, the filament may be extended radially outward to push against one or more tissue structures to direct the distal end of the sheath toward the target DRG, such as along a nerve root sleeve angulation. Such steps may optimize positioning of the sheath and any associated surgical tool or medical implant placed therein.

It may also be appreciated that in place of the filament, other structures and elements may instead be used. For example, a folded fan like structure folded flat against the sheath or a balloon configured to inflate and expand in a desired direction may instead be coupled to the sheath to articulate the sheath.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An introducer sheath comprising:
a tubular body having a proximal portion, a distal portion, and a central lumen, wherein the tubular body is configured for passage through a cannula; and
a filament constrained on or within at least a portion of the proximal portion, unconstrained over at least a portion of the distal portion, and constrained near a distal end of the tubular body,
wherein movement of the filament relative to the tubular body causes the unconstrained portion of the filament to extend radially outward from the tubular body so as to apply force to the distal end of the tubular body.

2. The introducer sheath of claim 1, wherein the cannula comprises an epidural needle and the tubular body is configured for advancement within an epidural space.

3. The introducer sheath of claim 2, wherein the application of force to the distal end of the tubular body repositions the distal end of the tubular body.

4. The introducer sheath of claim 2, wherein movement of the filament relative to the tubular body causes the unconstrained portion of the filament to extend radially outward so that the filament contacts a wall of the epidural space.

5. The introducer sheath of claim 2, wherein the tubular body is configured for advancement within the epidural space due to sufficient stiffness.

6. The introducer sheath of claim 5, wherein extension radially outward of the filament increases the stiffness.

7. The introducer sheath of claim 1, wherein the distal portion of the tubular body has a pre-curvature.

8. The introducer sheath of claim 7, wherein the pre-curvature has an angle in the range of approximately 80 to 165 degrees.

9. The introducer sheath of claim 7, wherein the unconstrained portion of the filament is disposed along the pre-curvature so that the filament extends radially outward from an outside curvature of the pre-curvature.

10. The introducer sheath of claim 1, wherein the distal end of the filament is removably attached near the distal end of the tubular body.

11. The introducer sheath of claim 1, wherein the tubular body has a side lumen extending at least partially through the tubular body and ending at a distal stop within which the filament is positioned.

12. The introducer sheath of claim 11, wherein the filament can be removably withdrawn from the side lumen of the tubular body.

13. The introducer sheath of claim 11, wherein the side lumen has a lateral opening through which the unconstrained portion of the filament is configured to extend radially outward. The introducer sheath of claim 13, wherein the lateral opening is disposed on the curved distal portion of the tubular body.

15. The introducer sheath of claim 14, wherein the curvature is curved in a first direction and the unconstrained portion of the filament is configured to extend radially outward in a second direction opposite the first direction.

16. The introducer sheath of claim 1, wherein tubular body has a low profile of 13 to 14 gauge or greater.

17. The introducer sheath of claim 1, wherein the tubular body is comprised of polyimide or polyetheretherketone.

18. The introducer sheath of claim 1, wherein the filament is comprised of stainless steel, surgical steel, or nitinol.

19. The introducer sheath of claim 1, further comprising a switch coupled to the proximal portion of the body and to the filament for articulating the filament between a radially extended configuration and a radially retracted configuration.

20. An introducer sheath comprising:
a tubular body having a proximal portion, a distal portion and a central lumen, wherein the tubular body is configured for passage through a cannula; and
a filament constrained on or within at least a portion of the proximal portion and unconstrained over at least a portion of the distal portion,
wherein movement of the filament relative to the tubular body causes the unconstrained portion of the filament to extend radially outward from the tubular body so as to engage a body tissue in a manner which assists in steering the tubular body within an area of a body.

21. The introducer sheath of claim 20, wherein a distal end of the filament is removably attached near a distal end of the tubular body.

22. The introducer sheath of claim 20, wherein the distal portion of the tubular body has curvature having an angle in the range of approximately 80 to 165 degrees.

23. The introducer sheath of claim 20, wherein the tubular body has a side lumen extending at least partially through the tubular body and ending at a distal stop.

24. The introducer sheath of claim 23, wherein the filament is positionable within the side lumen and can be removably withdrawn from the side lumen of the tubular body.

25. The introducer sheath of claim 23, wherein the filament is disposed within the side lumen of the tubular body and the distal stop is configured to limit advancement of the filament relative to the tubular body.

26. The introducer sheath of claim 23, wherein the side lumen has a lateral opening through which the unconstrained portion of the filament is configured to extend radially outward.

27. The introducer sheath of claim 26, wherein the lateral opening is disposed on a curvature of the distal portion of the tubular body.

28. The introducer sheath of claim 20, wherein the distal portion of the tubular body is curved in a first direction and the unconstrained portion of the filament is configured to extend radially outward in a second direction opposite the first direction.

29. The introducer sheath of claim 20, wherein the tubular body has a stiffness of approximately 0.10 lbs·in² to approximately 0.50 lbs·in².

30. The introducer sheath of claim 20, wherein tubular body has a low profile of 13 to 14 gauge or greater.

31. The introducer sheath of claim 20, wherein the tubular body is comprised of polyimide or polyetheretherketone.

32. The introducer sheath of claim 20, wherein the filament is comprised of stainless steel, surgical steel, or nitinol.

33. The introducer sheath of claim 20, further comprising a switch coupled to the proximal portion of the body and to the filament for articulating the filament between a radially extended configuration and a radially retracted configuration.

34. The introducer sheath of claim 20, wherein the body tissue comprises a wall of an epidural space and the area of the body comprises the epidural space.

35. A system for positioning an electrical stimulation lead near a target tissue, the system comprising:
the introducer sheath of claim 1 or 20; and
a lead comprising a shaft having at least one electrode disposed thereon, the lead being adapted to be advanced through the central lumen of the sheath.

36. The system of claim 35, wherein the sheath has an outer diameter which allows advancement through an introducer needle into a surgical space in a body of a patient and a stiffness which allows advancement along the surgical space to a position wherein the sheath can direct the lead toward the target tissue, and wherein withdrawal of the sheath positions the lead near the target tissue.

37. The system of claim 36, wherein the surgical space comprises an epidural space of a spinal column of the patient and the target tissue comprises a spinal nerve.

38. The system of claim 37, wherein the spinal nerve comprises a dorsal root ganglion (DRG).

39. A method for accessing a target tissue, the method comprising:
inserting an introducer sheath into a cannula accessing a surgical space in a body of a patient, wherein the sheath is comprised of a tubular body having a proximal portion, a distal portion, and a central lumen, and the sheath has a filament constrained on or within at least a portion of the proximal portion, unconstrained over at least a portion of the distal portion, and constrained near a distal end of the tubular body; and
moving the filament relative to the tubular body so that the unconstrained portion of the filament extends radially outward from the tubular body so as to assist in guiding the distal end of the tubular body toward the target tissue.

40. The method of claim 39, wherein the surgical space comprises an epidural space of the patient and the target tissue.

41. The method of claim 40, wherein the target tissue comprises a dorsal root ganglion.

42. The method of claim 40, further comprising moving the filament so that the unconstrained portion contacts a wall of the epidural space.

43. The method of claim 39, wherein the cannula comprises an introducing needle having an inner diameter of less than or equal to approximately 0.067 inches.

44. The method of claim 39, further comprising placing a lead into the sheath such that the lead extends out of the distal end of the sheath toward the target tissue.

45. The method of claim 44, further comprising positioning the sheath within the surgical space so the lead extends out of the distal end of the sheath toward a dorsal root ganglion.

46. The method of claim 39, further comprising withdrawing the filament from the tubular shaft before withdrawing the sheath from the surgical space.
